(19) United States
(12) Patent Application Publication
(10) Pub. No.: US 2026/0272411 A1
(43) Pub. Date: Sep. 17, 2026

(54) MEDICAL IMAGING METHOD AND MEDICAL IMAGING DEVICE

(71) Applicant: GE Precision Healthcare LLC, Waukesha, WI (US)

(72) Inventors: Qing Cao, Wuxi (CN); Yuanlong Meng, Wuxi (CN); Xiaojuan Zhu, Wuxi (CN); Siying Wang, Wuxi (CN)

(21) Appl. No.: 19/565,258

(22) Filed: Mar. 12, 2026

(30) Foreign Application Priority Data
Mar. 13, 2025 (CN) 202510299795.9

(51) Int. Cl. A61B 8/08; A61B 8/00

(52) U.S. Cl. CPC A61B 8/0883; A61B 8/463; A61B 8/5207; A61B 8/5223

(57) ABSTRACT

A medical imaging method including performing acquisition on a section of the heart in chronological order to obtain multiple images in two-dimensional mode; identifying at least one anatomical structure of interest in the multiple images in two-dimensional mode and determining a coordinate point of the at least one anatomical structure of interest; generating at least one virtual sampling line in the multiple images in two-dimensional mode, the at least one virtual sampling line passing through the at least one anatomical structure of interest; generating a displacement time map of the at least one anatomical structure of interest by using the at least one virtual sampling line on the basis of the multiple images in two-dimensional mode; and detecting multiple corresponding sets of end-systolic coordinates and end-diastolic coordinates on the basis of the displacement time map to determine multiple cardiac cycles.

### CROSS-REFERENCE TO RELATED APPLICATION

This application claims priority to Chinese Patent Application No. 202510299795.9, which was file on March 13, 2025 at the Chinese Patent Office. The entire contents of the above-listed application are incorporated by reference herein in their entirety.

### TECHNICAL FIELD

The present disclosure generally relates to the medical field, and more particularly, to a medical imaging method and a medical imaging apparatus for obtaining end-diastole and end-systole through real-time simulation on an image in two-dimensional mode, and calculating a cardiac cycle.

### BACKGROUND ART

In the current medical field, when it is necessary to know about the structural form of an internal organ of the human body and the heart rate status, an ultrasound device is often used to generate, in a mode such as B-mode, M-mode, or anatomic M-mode, a two-dimensional image for the internal organ, so as to examine the structural form of the internal organ and the heart rate status.

Two-dimensional ultrasound imaging techniques are widely used in cardiac ultrasound examination. Generally, two-dimensional ultrasound imaging mainly comprises observing a heart chamber, the internal diameter of a blood vessel, and the thickness of a heart valve on the basis of a two-dimensional image obtained by performing ultrasound scanning in B-mode, and observing the heart rate status of a mitral valve, a tricuspid valve, an aortic valve, a pulmonary valve, etc. on the basis of a two-dimensional image obtained by performing ultrasound scanning in M-mode or anatomic M-mode. In some cases, displaying information related to a cardiac cycle while a two-dimensional ultrasound image is being observed can help an examiner to more accurately perform heart disease diagnosis.

Under existing technical conditions, in order to display an electrocardiogram or information related to a cardiac cycle while displaying a two-dimensional ultrasound image, an examiner generally needs to use an external electrocardiograph to obtain an electrocardiogram signal, which will complicate the operation of the examiner. Alternatively, the ultrasound device may be switched to an M-mode or an anatomic M-mode (AMM) so as to detect a cardiac cycle. However, this will sacrifice the resolution of a two-dimensional image obtained in B-mode.

Therefore, there is a need for a technique that can display a cardiac cycle in a simpler and less power-consuming manner so as to help an examiner to more efficiently and accurately perform heart disease diagnosis.

### SUMMARY

The present disclosure aims to overcome the above and/or other problems in the prior art. According to the present disclosure, a medical imaging method and a medical imaging device for obtaining a cardiac cycle through real-time simulation on an image in two-dimensional mode is provided. The method and the device allow performing simulation processing on the image in two-dimensional mode to obtain the cardiac cycle, without both the need for an external electrocardiograph and the need to switch to M-mode or anatomic M-mode, thereby helping an examiner to more conveniently, more efficiently, and more accurately perform heart disease diagnosis.

A first aspect of the present disclosure provides a medical imaging method, which can comprise the following steps: performing acquisition on a section of the heart in chronological order to obtain multiple images in two-dimensional mode; identifying at least one anatomical structure of interest in the multiple images in two-dimensional mode and determining a coordinate point of the at least one anatomical structure of interest; generating at least one virtual sampling line in the multiple images in two-dimensional mode, the at least one virtual sampling line passing through the at least one anatomical structure of interest; generating a displacement time map of the at least one anatomical structure of interest by using the at least one virtual sampling line on the basis of the multiple images in two-dimensional mode; and detecting multiple corresponding sets of end-systolic coordinates and end-diastolic coordinates on the basis of the displacement time map to determine multiple cardiac cycles.

As described above, the medical imaging method of the first aspect of the present disclosure innovatively introduces virtual cursor lines so as to transform analysis of an image in two-dimensional mode into analysis of an image in a different mode. As a result, the need to develop multiple models for different scanning views is avoided, and the detection of a cardiac cycle is implemented through a unified method, thereby greatly reducing system complexity, computational burden, and resource consumption.

In the medical imaging method of the first aspect of the present disclosure, in order to make it convenient for an examiner to perform diagnosis, the multiple images in two-dimensional mode or a video in two-dimensional mode consisting of the multiple images in two-dimensional mode are/is displayed, and at the same time an image of end-systole and an image of end-diastole in each of the multiple cardiac cycles and a cardiac cycle indication are displayed.

As described above, the medical imaging method of the first aspect of the present disclosure can allow displaying a cardiac cycle indication obtained by automatic detection through real-time simulation of a electrocardiogram while displaying an image in two-dimensional mode or a video in two-dimensional mode, without both the need for an external electrocardiograph and the need to switch to an M-mode or an anatomic M-mode. Therefore, an examiner can observe the feature of a cardiac structure and the feature of a cardiac cycle at the same time without being interfered with when performing scanning, thereby improving the efficiency and accuracy in heart disease diagnosis by the examiner.

Further, in the foregoing medical imaging method, in response to an instruction from an input unit, one cardiac cycle is selected from the multiple cardiac cycles as a target cardiac cycle, the image of end-systole and the image of end-diastole corresponding to the target cardiac cycle are displayed in a highlighted manner, and the target cardiac cycle is displayed in a highlighted manner in the cardiac cycle indication.

As described above, the medical imaging method of the first aspect of the present disclosure allows an examiner to select and display in a highlighted manner as needed, through an interactive user interface, an image of end-systole ES and an image of end-diastole ED as a set, and at the same time, display in a highlighted manner a corresponding cardiac cycle in the cardiac cycle indication without displaying other cardiac cycles, thereby enhancing the convenience in heart disease diagnosis by the examiner.

In the medical imaging method of the first aspect of the present disclosure, it may also be that the at least one virtual sampling line comprises multiple virtual sampling lines, and the generating a displacement time map of the at least one anatomical structure of interest comprises: using the coordinate point of the at least one anatomical structure of interest as a center point to generate the multiple virtual sampling lines passing through the center point; and generating multiple displacement time maps as described on the basis of the multiple virtual sampling lines, and the determining multiple cardiac cycles comprises: stacking multiple displacement time maps as described along the Y-axis to obtain a second composite displacement time map; and detecting multiple corresponding sets of the end-systolic coordinates and the end-diastolic coordinates on the basis of the second composite displacement time map to determine the multiple cardiac cycles.

As described above, the medical imaging method of the first aspect of the present disclosure can generate multiple different virtual sampling lines in a section of the heart. Therefore, the end-systolic coordinates and the end-diastolic coordinates can be determined by acquiring, from different angles, the amounts of displacement of the same position of the anatomical structure of interest at different time points by using the different virtual sampling lines, and thus the accuracy in the detection of a cardiac cycle can also be improved.

In the medical imaging method of the first aspect of the present disclosure, it may be that the at least one anatomical structure of interest comprises multiple anatomical structures of interest, and the generating a displacement time map of the at least one anatomical structure of interest comprises: generating the at least one virtual sampling line for each of the multiple anatomical structures of interest, respectively; and generating multiple displacement time maps as described on the basis of the multiple generated virtual sampling lines as described, and determining multiple cardiac cycles comprises: stacking multiple displacement time maps as described along the Y-axis to obtain a first composite displacement time map; and detecting multiple corresponding sets of the end-systolic coordinates and the end-diastolic coordinates on the basis of the first composite displacement time map to determine the multiple cardiac cycles.

As described above, the medical imaging method of the first aspect of the present disclosure can determine multiple different anatomical structures of interest in a section of the heart. Therefore, the end-systolic coordinates and the end-diastolic coordinates can be determined by using pixel changes of the different anatomical structures of interest at the same time point, and thus the accuracy in the detection of a cardiac cycle can be improved.

In the medical imaging method of the first aspect of the present disclosure, the at least one anatomical structure of interest is identified using a first neural network model.

Further, in the foregoing medical imaging method, the multiple cardiac cycles are determined using a second neural network model different from the first neural network model.

Still further, in the foregoing medical imaging method, the second neural network model only predicts an X-axis coordinate, and the multiple corresponding sets of the end-systolic coordinates and the end-diastolic coordinates are detected by processing the X-axis coordinate.

As described above, the medical imaging method of the first aspect of the present disclosure does not need to utilize all of the computational functions of the second neural network model when detecting the end-systolic coordinates and the end-diastolic coordinates, but only needs to utilize the computational function of the second neural network model for the X-coordinate. Therefore, the complexity, computational burden, and resource consumption of the computational model can be further reduced.

In the medical imaging method of the first aspect of the present disclosure, the multiple images in two-dimensional mode are any one of an image in B-mode, an image in M-mode, or an image in anatomic M-mode.

As described above, the medical imaging method of the first aspect of the present disclosure makes it possible that any image in two-dimensional mode that can be obtained by means of a two-dimensional ultrasound device can be applicable to the medical imaging method of the present disclosure. Therefore, the usage of an existing two-dimensional ultrasound device can be increased, thereby improving the utility value of the two dimensional ultrasound device.

Yet further, in the medical imaging method of the first aspect of the present disclosure, the section and the at least one anatomical structure of interest are selected from at least one of the following schemes: the section is a two-chamber cardiac section, and the at least one anatomical structure of interest comprises a mitral valve; the section is a three-chamber cardiac section, and the at least one anatomical structure of interest comprises a mitral valve or an aortic valve; the section is a four-chamber cardiac section, and the at least one anatomical structure of interest comprises a mitral valve or a tricuspid valve; the section is a five-chamber cardiac section, and the at least one anatomical structure of interest comprises a mitral valve and a tricuspid valve; the section is a parasternal long-axis section, and the at least one anatomical structure of interest comprises a mitral valve or an aortic valve; or the section is a short-axis section, and the at least one anatomical structure of interest comprises an aortic valve.

As described above, according to the medical imaging method of the first aspect of the present disclosure, data acquisition performed on any section of the heart would be applicable to the medical imaging method of the present disclosure. Therefore, the efficiency of heart disease diagnosis by an examiner can be further improved, and the complexity of the operation can be reduced.

According to a second aspect of the present disclosure, a medical imaging system is provided, comprising a processor configured to execute the foregoing ultrasound imaging method as described in the first aspect of the present disclosure.

According to a third aspect of the present disclosure, a computer-readable storage medium is provided, having coded instructions recorded thereon such that when the instructions are executed, the foregoing medical imaging method as described in the first aspect of the present disclosure is implemented.

Other features and aspects of the present disclosure will become clearer via the detailed description provided below with reference to the accompanying drawings.

### BRIEF DESCRIPTION OF THE DRAWINGS

The present disclosure can be better understood by means of the description of the exemplary embodiments of the present disclosure in conjunction with the drawings, in which:

FIG. 1 is a flowchart showing generation of a medical image by using the medical imaging method according to an embodiment of the present disclosure;

FIG. 2 is a flowchart showing displaying of a medical image by using the medical imaging method according to an embodiment of the present disclosure;

FIG. 3 is an overall schematic diagram showing the medical imaging method of a first example of an embodiment of the present disclosure;

FIG. 4 is a partially enlarged schematic diagram showing step b1 and step c1 in the medical imaging method of the first example of an embodiment of the present disclosure;

FIG. 5 is a partially enlarged schematic diagram showing step d1 in the medical imaging method of the first example of an embodiment of the present disclosure;

FIG. 6 is a partially enlarged schematic diagram showing step e1 in the medical imaging method of the first example of an embodiment of the present disclosure;

FIG. 7 is a partially enlarged schematic diagram showing step b2 and step c2 in the medical imaging method of a second example of an embodiment of the present disclosure;

FIG. 8 is a partially enlarged schematic diagram showing step d2 in the medical imaging method of the first example of an embodiment of the present disclosure;

FIG. 9 shows a display screen A1 corresponding to step G in the medical imaging method of an embodiment of the present disclosure;

FIG. 10 shows a display screen A2 corresponding to step G in the medical imaging method of an embodiment of the present disclosure;

FIG. 11 is a diagram showing a comparison between a case where a determined cardiac cycle is displayed in B-mode using the medical imaging method according to an embodiment of the present disclosure and a case where a determined cardiac cycle is displayed in M-mode or anatomic M-mode in the prior art.

### DETAILED DESCRIPTION

The present disclosure will be further described below with reference to specific embodiments and the accompanying drawings. More details are set forth in the following description in order to facilitate thorough understanding of the present disclosure, but it will be apparent that the present disclosure can be implemented in many other forms other than those described herein, and those skilled in the art can, without departing from the spirit of the present disclosure, make similar alterations and modifications according to practical applications. Therefore, the scope of protection of the present disclosure should not be limited by the contents of the specific embodiments.

Unless defined otherwise, technical terms or scientific terms used in the claims and description should have the usual meanings that are understood by those of ordinary skill in the technical field to which the present disclosure belongs. Terms such as “first”, “second”, and similar terms used in the description and claims of the present application do not denote any order, quantity, or importance, but are only intended to distinguish different constituents. The terms “one” or “a/an” and similar terms do not express a limitation of quantity, but rather that at least one is present. The terms “include” or “comprise” and similar words indicate that an element or object preceding the terms “include” or “comprise” encompasses elements or objects and equivalent elements thereof listed after the terms “include” or “comprise”, and do not exclude other elements or objects.

According to an embodiment of the present disclosure, a medical imaging method for obtaining a cardiac cycle through real-time simulation on an image in two-dimensional mode is described in detail.

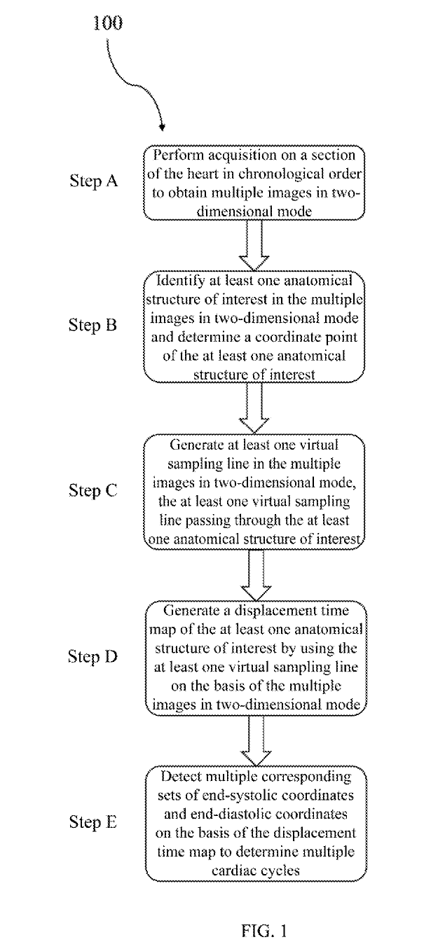

An overall process of the medical imaging method according to an embodiment of the present disclosure will be explained below with reference to FIG. 1.

FIG. 1 is a flowchart showing generation of a medical image by the medical imaging method according to an embodiment of the present disclosure. As shown in FIG. 1, the medical imaging method 100 may comprise step A, step B, step C, step D, and step E.

In step A, a section of the heart is selected, and this section of the heart is imaged using a two-dimensional ultrasound device, that is, acquisition is performed on the section of the heart in chronological order, thereby obtaining multiple images in two-dimensional mode in chronological order corresponding to the section of the heart.

When selecting a section of the heart, there is no limitation on which section of the heart is to be selected. For example, any one of a parasternal long-axis section (PLAX), an apical four-chamber cardiac section (A4CH), an apical two-chamber cardiac section (A2CH), etc. may be selected.

In step B, for the multiple images in two-dimensional mode obtained in step A above, one or more cardiac structures are identified as at least one anatomical structure of interest, and then, for the at least one anatomical structure of interest, all coordinate points thereof are acquired.

The at least one anatomical structure of interest may be one anatomical structure of interest, or may be multiple anatomical structures of interest. In addition, the one or more anatomical structures of interest may be selected from among a mitral valve, a tricuspid valve, an aortic valve, a pulmonary valve, etc. of the cardiac structure.

Preferably, in the case of selecting a section of the heart and an anatomical structure of interest therein, when the section is a two-chamber cardiac section, the anatomical structure of interest may include a mitral valve; when the section is a three-chamber cardiac section, the anatomical structure of interest may include a mitral valve or an aortic valve; when the section is a four-chamber cardiac section, the anatomical structure of interest may include a mitral valve or a tricuspid valve; when the section is a five-chamber cardiac section, the anatomical structure of interest may include a mitral valve and a tricuspid valve; when the section is a parasternal long-axis section, the anatomical structure of interest may include a mitral valve or an aortic valve; and, when the section is a short-axis section, the anatomical structure of interest may include an aortic valve. Any of the various cases described above is applicable to the present disclosure.

In step C, in the multiple images in two-dimensional mode obtained in step A above, at least one virtual sampling line passing through the anatomical structure of interest is generated for each of the at least one anatomical structure of interest obtained in step B above, such that at least one virtual sampling line is generated at the coordinate point of each anatomical structure of interest.

The at least one virtual sampling line may be one virtual sampling line or multiple virtual sampling lines. In addition, there is no particular limitation on the way of generating the virtual sampling line, as long as the virtual sampling line passes through the anatomical structure of interest.

In step D, on the basis of the multiple images in two-dimensional mode obtained in step A above, the amounts of positional change generated in chronological order by the at least one anatomical structure of interest obtained in step B above are acquired by using the at least one virtual sampling line obtained in step C above, thereby obtaining a displacement time map of the at least one anatomical structure of interest.

With regard to the displacement time map above, since the at least one anatomical structure of interest undergoes displacement at different time points, the displacement time map above is a map representing the amounts of positional change of the at least one anatomical structure of interest in chronological order.

In step E, on the basis of the displacement time map obtained in step D above, multiple corresponding sets of end-systolic coordinates and end-diastolic coordinates are detected in the multiple images in two-dimensional mode obtained in step A above, and then multiple cardiac cycles are determined according to the multiple corresponding sets of end-systolic coordinates and end-diastolic coordinates.

Each of the cardiac cycles is mainly determined according to a positional relationship between the end-systolic coordinates and end-diastolic coordinates of a corresponding set.

As described above, the medical imaging method 100 according to an embodiment of the present disclosure involves determining at least one anatomical structure of interest and generating at least one virtual sampling line, so that analysis of an image in two-dimensional mode is transformed into analysis of an image in a different mode. As a result, the need to develop multiple models for different scan views is avoided, and the detection of a cardiac cycle is implemented through a unified method, thereby greatly reducing system complexity, computational burden, and resource consumption. Moreover, the medical imaging method according to an embodiment of the present disclosure can allow displaying a cardiac cycle indication obtained by automatic detection through real-time simulation of a electrocardiogram while displaying an image in two-dimensional mode or a video in two-dimensional mode, without both the need for an external electrocardiograph and the need to switch to an M-mode or an anatomic M-mode. Therefore, an examiner can observe the feature of a cardiac structure and the feature of a cardiac cycle at the same time without being interfered with when performing scanning, thereby improving the efficiency and accuracy in heart disease diagnosis by the examiner.

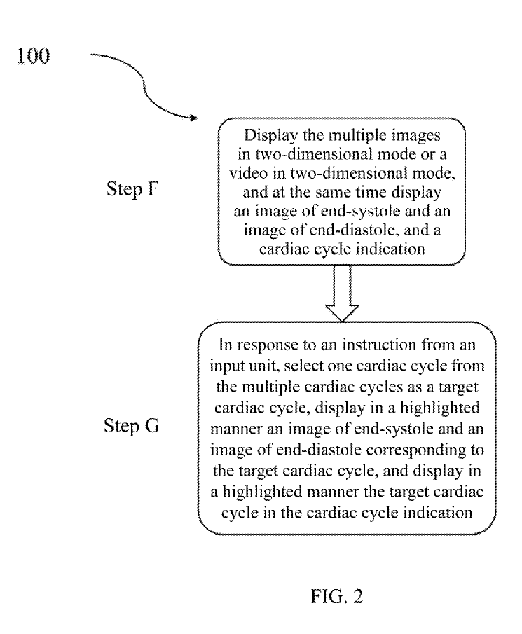

An overall process of displaying a medical image by the medical imaging method 100 according to an embodiment of the present disclosure will be described below with reference to FIG. 2.

Firstly, FIG. 2 is a flowchart showing displaying of a medical image by using the medical imaging method according to an embodiment of the present disclosure. As shown in FIG. 2, the medical imaging method 100 further comprises step F and step G.

As shown in FIG. 2, in step F, the multiple images in two-dimensional mode of an apical four-chamber cardiac section obtained in step A above, or a video in two-dimensional mode consisting of the multiple images in two-dimensional mode of the apical four-chamber cardiac section, are displayed on a display screen of an ultrasound device. At the same time, also displayed on the display screen are an image of end-systole ES and an image of end-diastole ED of each of the multiple cardiac cycles obtained in step E above, and further displayed on the display screen is a cardiac cycle indication that includes each cardiac cycle corresponding to the end-systole ES and the end-diastole ED in each cardiac cycle above.

By the cardiac cycle indication above it is meant obtaining an image that represents a cardiac cycle by simulating in real time an electrocardiogram signal obtained by, for example, an electrocardiograph (ECG).

The display positions of the multiple images in two-dimensional mode or the video in two-dimensional mode, the image of end-systole ES and the image of end-diastole ED, and multiple cardiac cycles indications are not limited to the case described above, so long as the way of displaying is convenient for an examiner to perform observation.

Next, as shown in FIG. 2, in step G, when the examiner needs to select one cardiac cycle from the multiple cardiac cycles as a target cardiac cycle for observation, the examiner may input an instruction into the ultrasound device via an input unit. Upon receiving the instruction from the input unit, the ultrasound device, in response to the instruction, displays in a highlighted manner on the display screen, a set of the image of end-systole ES and the image of end-diastole ED corresponding to the target cardiac cycle, and displays in a highlighted manner the target cardiac cycle in the cardiac cycle indication.

The input unit may be a touch panel, a knob, a keyboard, etc. configured on the ultrasound device, or may be an external apparatus configured separately from the ultrasound apparatus.

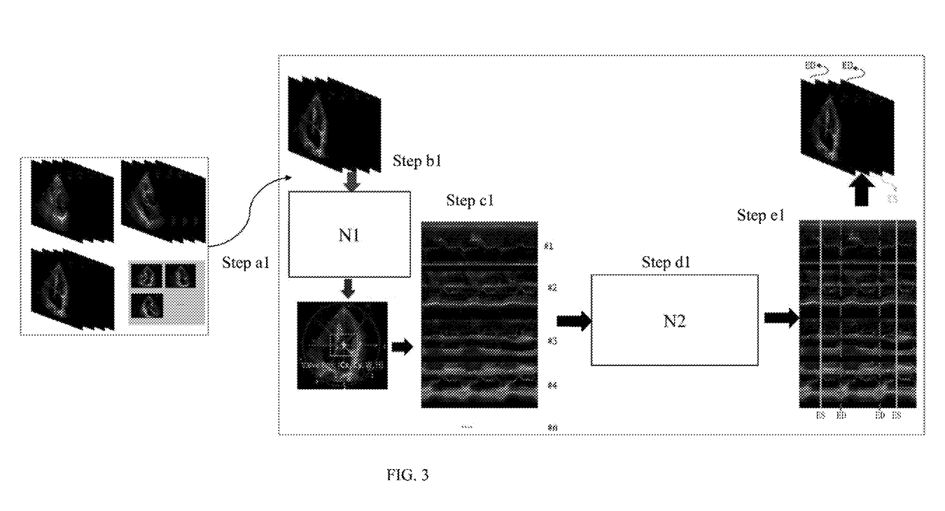

A specific implementation of the medical imaging method of a first example of an embodiment of the present disclosure will be described below with reference to FIGS. 3 to 6.

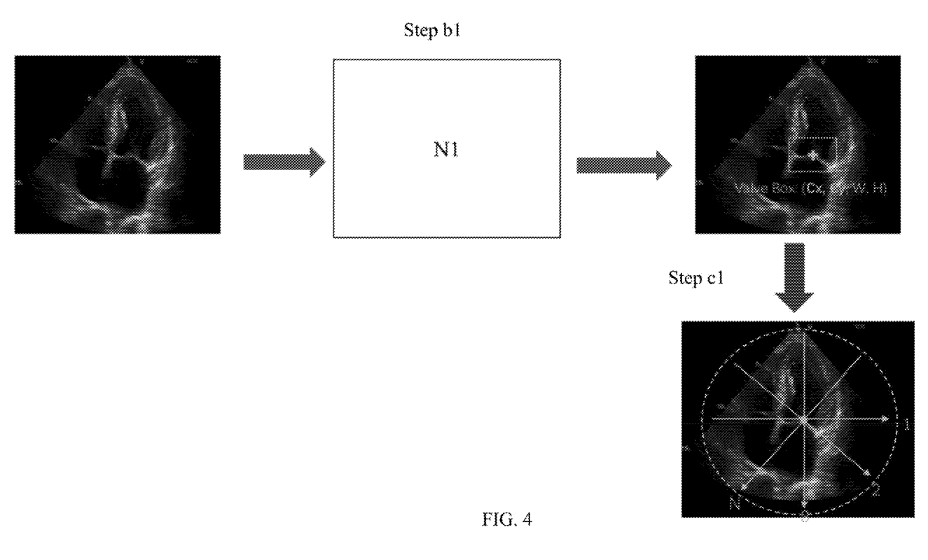

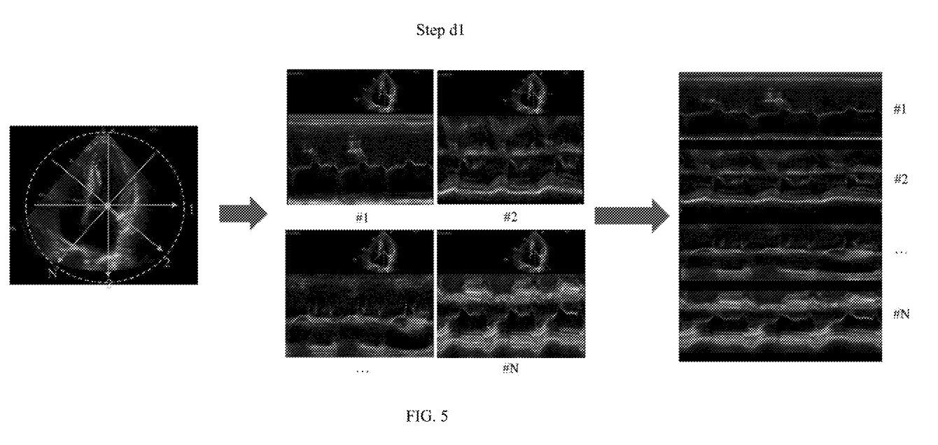

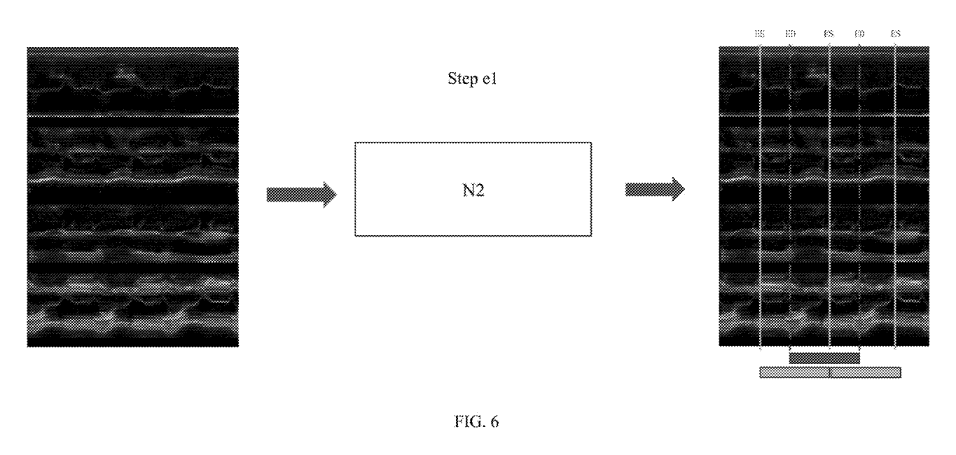

FIG. 3 is an overall schematic diagram showing the medical imaging method of the first example of an embodiment of the present disclosure; FIG. 4 is a partially enlarged schematic diagram showing step b1 and step c1 in the medical imaging method of the first example of an embodiment of the present disclosure; FIG. 5 is a partially enlarged schematic diagram showing step d1 in the medical imaging method of the first example of an embodiment of the present disclosure; and FIG. 6 is a partially enlarged schematic diagram showing step e1 in the medical imaging method of the first example of an embodiment of the present disclosure. Step b1, step c1, step d1, and step e1 shown in FIGS. 3 to 6 represent a specific implementation of step A, step B, step C, step D, and step E above, respectively.

In the first example of an embodiment of the present disclosure, as shown in FIGS. 3 to 6, an image in B-mode is used as an example to explain the medical imaging method for an image in two-dimensional mode. The present disclosure is not limited to the image in B-mode, and an image in M-mode or anatomic M-mode, etc. may also be used. So long as the image is one in two-dimensional mode, it is applicable to the present disclosure.

First, as shown in FIG. 3, in step a1, the ultrasound device is set to B-mode, and then the entire structure of the heart is scanned from different sectioning angles. The scanning results within a certain time period are taken as multiple sets of B-mode echocardiogram in different scan views, each set of B-mode echocardiogram having at least one image in two-dimensional mode. Then, any set of B-mode echocardiogram among the multiple sets of B-mode echocardiogram is selected as a B-mode echocardiogram to be imaged.

With regard to the different sectioning angles, in the first example of an embodiment of the present disclosure, a total of three sets of B-type echocardiogram for three sections, namely, a parasternal long-axis section (PLAX), an apical four-chamber cardiac section (A4CH), and an apical two-chamber cardiac section (A2CH), are obtained by scanning. However, there is no particular limitation in the present disclosure on the selection of the sectioning angles. Further, in the first example of an embodiment of the present disclosure, a set of B-mode echocardiograms for the apical four-chamber cardiac section (A4CH) obtained by performing scanning on the selected apical four-chamber cardiac section (A4CH) is taken as an example for explanation.

Next, as shown in FIGS. 3 and 4, in step b1, for the B-mode echocardiogram of the selected apical four-chamber cardiac section (A4CH), one anatomical structure is selected as an anatomical structure of interest Valve. Then, the positional coordinates of the position of this anatomical structure of interest Valve is predicted using an artificial neural network model N1. It can be understood that the artificial neural network model N1 may be selected from any one in the prior art, e.g., FasterRCNN, and there is no limitation in the present disclosure in this respect.

In the first example of an embodiment of the present disclosure, in the case of selecting the apical four-chamber cardiac section (A4CH), it is preferable to select the mitral valve anatomical structure as the anatomical structure of interest Valve.

Specifically, using the artificial neural network model N1, one selection box is generated on each image in two-dimensional mode in the B-mode echocardiogram for the apical four-chamber cardiac section (A4CH). The center point of the selection box is the coordinates Valve Box (Cx, Cy, W, H) of the position of the mitral valve anatomical structure, wherein Cx represents the horizontal axis coordinate, Cy represents the vertical axis coordinate, W represents the width of the selection box, and H represents the height of the selection box.

With regard to the selection box described above, the coordinates Valve Box (Cx, Cy, W, H) of the center point represents a position having the highest probability of being predicted to be the mitral valve anatomical structure through recognition, and the selection box formed by the width W and the height H represents a position having a relatively high probability of being predicted to be the mitral valve anatomical structure through recognition and determination. Therefore, in the present disclosure, the position indicated by the coordinates Valve Box (Cx, Cy, W, H) of the center point of the selection box is used as the position of the mitral valve anatomical structure in the B-mode echocardiogram.

In addition, in the first example of an embodiment of the present disclosure, using the artificial neural network model N1 to predict the coordinate point of the position of the mitral valve anatomical structure can not only accurately predict the position of the mitral valve anatomical structure in the B-mode echocardiogram, but also can reduce overall computational burden. It can be understood that any artificial neural network model N1 that can accurately predict the coordinate point of the mitral valve anatomical structure can be applied to the medical imaging method of the first example of an embodiment of the present disclosure.

In addition, as shown in FIGS. 3 and 4, in step c1, for each image in two-dimensional mode in the B-mode echocardiogram for the apical four-chamber cardiac section (A4CH), the coordinates Valve Box (Cx, Cy, W, H) of the predicted mitral valve anatomical structure are used as an intersection point to generate N virtual sampling lines passing through the intersection point. As shown in FIG. 3, in the first example of an embodiment of the present disclosure, the N virtual sampling lines have the same length, and the middle point of each of the virtual sampling lines is made to coincide with the coordinate point Valve Box (Cx, Cy, W, H) of the predicted mitral valve anatomical structure, although a non-middle point of any of the N virtual sampling lines may be made to coincide with the coordinate point Valve Box (Cx, Cy, W, H) of the predicted mitral valve anatomical structure.

Then, as shown in FIGS. 3 and 5, in step d1, for each of the N virtual sampling lines, the amounts of positional change, occurring within a predetermined time period, at the position of the heart anatomical structure corresponding to each virtual sampling line are detected according to all the images in two-dimensional mode in the B-mode echocardiogram for the apical four-chamber cardiac section (A4CH), thereby obtaining N displacement time maps #1, #2, #3, #4, ..., #N shown in FIG. 5.

Next, with the time as the X axis and the amount of displacement as the Y axis, the N displacement time maps are stacked along the Y axis in a manner of being aligned with respect to the X axis, thereby obtaining a first composite displacement time map. Hence, the amounts of displacement of the same position of the mitral valve anatomical structure occurring at different time points and the locations of different positions of the mitral valve anatomical structure at the same time point can be observed.

Finally, as shown in FIGS. 3 and 6, in step e1, the end-diastolic coordinates ED and the end-systolic coordinates ES of the mitral valve anatomical structure in chronological order are detected according to the above-obtained first composite displacement time map by using the DETR neural network model N2. As shown in FIG. 6, in the first composite displacement time map, the end-diastolic (ED) coordinates are indicated by solid vertical lines, and the end-systolic (ES) coordinates are indicated by dashed vertical lines.

In addition, in the first example of an embodiment of the present disclosure, the use of the DETR neural network model N2 to detect the end-diastolic (ED) coordinates and the end-systolic (ES) coordinates of the mitral valve anatomical structure of the apical four-chamber cardiac section (A4CH) in chronological order can not only accurately predict end-diastole ED and end-systole ES in the B-mode echocardiogram, but also can reduce the overall computational burden of the neural network model.

Furthermore, preferably, the DETR neural network model N2 may be modified so that the model only needs to predict the X-axis coordinates (Cx, W) in order to be able to detect multiple corresponding sets of the end- systolic (ED) coordinates and the end- diastolic (ES) coordinates by processing the X-axis coordinates (Cx, W). Hence, the overall computational burden of the neural network model could be further reduced.

However, although the DETR neural network model N2 is used here as an example for explanation, any model that can accurately predict end-diastole ED and end-systole ES in the B-mode echocardiogram can be applied to the medical imaging method of the first example of an embodiment of the present disclosure.

As shown in FIGS. 3 and 6, on the basis of the above-obtained multiple sets of the end-diastolic (ED) coordinates and the end-systolic (ES) coordinates, the interval between an adjacent set of the end-diastolic (ED) coordinates and the end-systolic (ES) coordinates is determined as one cardiac cycle, thereby determining multiple cardiac cycles.

As described above, the medical imaging method of the first example of an embodiment of the present disclosure can generate multiple different virtual sampling lines for an anatomical structure of interest in a section of the heart. Therefore, the end-systolic (ES) coordinates and the end-diastolic (ED) coordinates can be determined by acquiring, from different angles, the amounts of displacement of the same position of the anatomical structure Valve of interest at different time points by using the multiple different virtual sampling lines, and thus the accuracy in the detection of a cardiac cycle can also be improved.

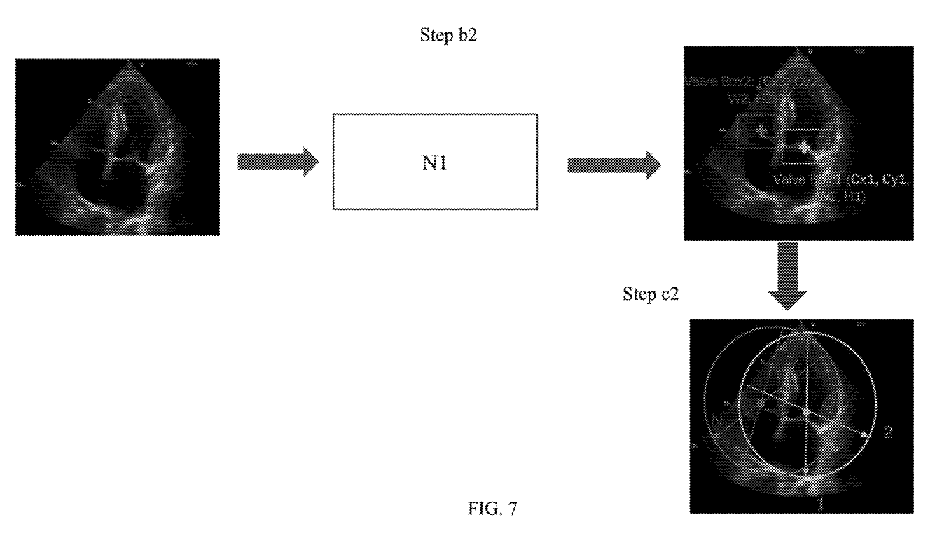

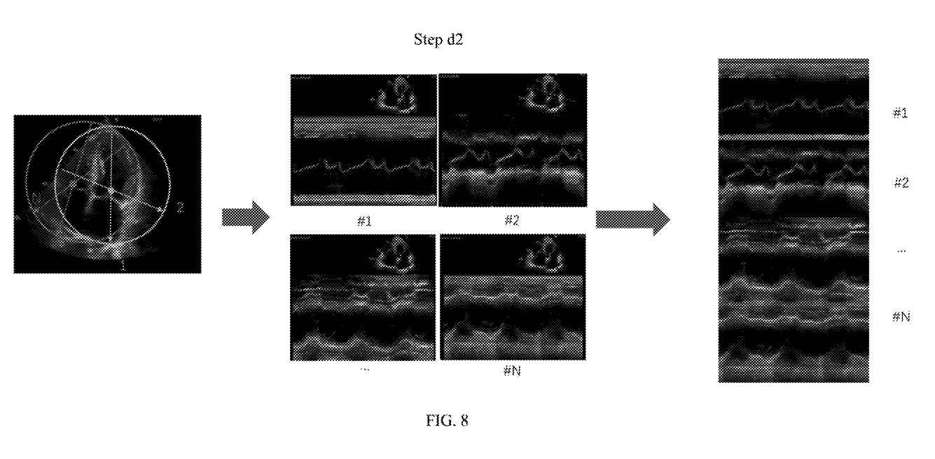

Another specific implementation of the medical imaging method of a second example of an embodiment of the present disclosure will be described below with reference to FIGS. 7 to 8.

FIG. 7 is a partially enlarged schematic diagram showing step b2 and step c2 in the medical imaging method of the second example of an embodiment of the present disclosure; and FIG. 8 is a partially enlarged schematic diagram showing step d2 in the medical imaging method of the first example of an embodiment of the present disclosure. Step b2, step c2, and step d2 shown in FIGS. 7 to 8 represent another specific implementation of step B, step C, and step D above, respectively.

In addition, although not shown, in the medical imaging method of the second example, like in the medical imaging method of the first example, the case where a set of B-mode echocardiograms for the apical four-chamber cardiac section (A4CH) is first obtained using step a1, and the end-systolic (ES) coordinates and the end-diastolic (ED) coordinates are finally determined using step e1 will be used as an example for explanation.

First, as shown in FIG. 7, in step b2, for the set of B-mode echocardiograms for the apical four-chamber cardiac section (A4CH), two anatomical structures are selected as a first anatomical structure of interest Valve1 and a second anatomical structure of interest Valve2, and then the positional coordinates of the respective positions of the first anatomical structure of interest Valve1 and the second anatomical structure of interest Valve2 are predicted by using an artificial neural network model N1.

In the second example of an embodiment of the present disclosure, in the case where the apical four-chamber cardiac section (A4CH) is selected, it is preferable to select a mitral valve anatomical structure as the first anatomical structure of interest Valve1, and at the same time select a tricuspid valve anatomical structure as the second anatomical structure of interest Valve2.

Specifically, using the artificial neural network model N1, two selection boxes are generated on each image in two-dimensional mode in the B-mode echocardiograms for the apical four-chamber cardiac section (A4CH). The center points of the two selection boxes are the coordinates Valve Box1 (Cx1, Cy1, W1, H1) of the position of the mitral valve anatomical structure and the coordinates Valve Box2 (Cx2, Cy2, W2, H2) of the position of the tricuspid valve anatomical structure, respectively, wherein Cx1 and Cx2 represent the horizontal axis coordinate, respectively, Cy1 and Cy2 represent the vertical axis coordinate, respectively, W1 and W2 represent the width of the selection boxes, respectively, and H1 and H2 represent the height of the selection boxes, respectively.

Then, as shown in FIG. 7, in step c2, for each image in two-dimensional mode in the B-mode echocardiograms for the apical four-chamber cardiac section (A4CH), the coordinates Valve Box1 (Cx1, Cy1, W1, H1) of the predicted mitral valve anatomical structure are used as an intersection point to generate at least one virtual sampling line passing through the intersection point, and the coordinates Valve Box2 (Cx2, Cy2, W2, H2) of the position of the predicted tricuspid valve anatomical structure are used as an intersection point to generate at least one virtual sampling line passing through the intersection point, thereby generating N virtual sampling lines in total.

Then, as shown in FIG. 8, in step d2, for each of the N virtual sampling lines, the amounts of positional change, occurring within a predetermined time period, at the position of the heart anatomical structure corresponding to each virtual sampling line is detected according to all the images in two-dimensional mode in the B-mode echocardiogram for the apical four-chamber cardiac section (A4CH), thereby obtaining N displacement time maps #1, #2, #3, #4, ..., #N shown in FIG. 8.

Next, with the time as the X axis and the amount of displacement as the Y axis, the N displacement time maps are stacked along the Y axis in a manner of being aligned with respect to the X axis, thereby obtaining a second composite displacement time map. Hence, the amounts of displacement of the same position of different anatomical structures of the heart occurring at different time points can be observed.

Finally, although not shown, in the medical imaging method of the second example, like in the medical imaging method of the first example, the end-diastolic (ED) coordinates and the end-systolic (ES) coordinates of the mitral valve anatomical structure in chronological order are detected according to the above-obtained second composite displacement time map by using the DETR neural network model N2, thereby determining multiple cardiac cycles.

In addition, the explanations with respect to the above-mentioned selection boxes, artificial neural network model N1, virtual sampling lines, and DETR neural network model N2 are similar to those for the first example, and are therefore omitted here to avoid redundancy.

As described above, the medical imaging method of the second example of an embodiment of the present disclosure can generate at least one different virtual sampling line for multiple anatomical structures of interest in a section of the heart. Therefore, the end-systolic (ES) coordinates and the end-diastolic (ED) coordinates can be determined by acquiring, from different angles, the amounts of displacement of the same position of the multiple anatomical structures of interest at different time points by using the multiple different virtual sampling lines, and thus the accuracy in the detection of a cardiac cycle can also be improved.

In summary of the above, various cases in which the medical imaging method according to an embodiment of the present disclosure comprises identifying at least one anatomical structure of interest and generating at least one virtual sampling line have been described with reference to FIGS. 3 to 8.

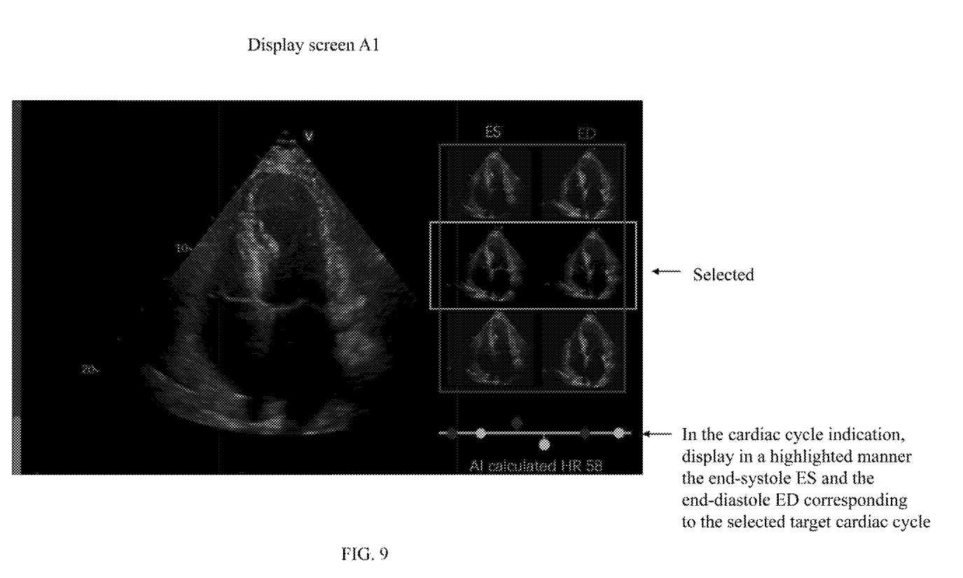

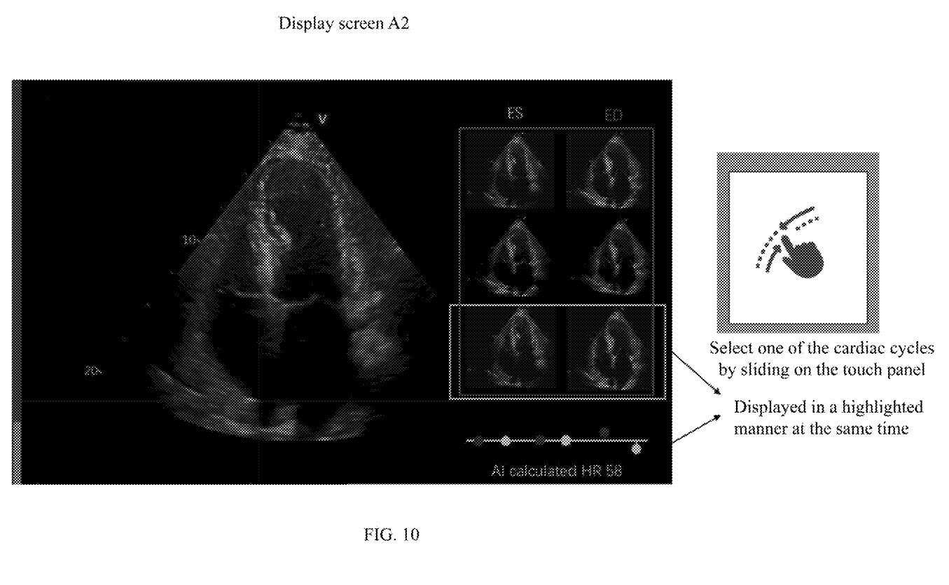

Below, the display screen corresponding to step G of the medical imaging method according to an embodiment of the present disclosure will be described with reference to FIGS. 9 to 10.

First, FIG. 9 shows a display screen A1 corresponding to step G in the medical imaging method of an embodiment of the present disclosure; and FIG. 10 shows a display screen A2 corresponding to step G in the medical imaging method of an embodiment of the present disclosure. As shown in FIG. 9, in the display screen A1 corresponding to step G above, an image in two-dimensional mode obtained by scanning and imaging a section of the heart using an ultrasound device or a video in two-dimensional mode consisting of the images in two-dimensional mode is displayed on the left part, an image of end-systole ES and an image of end-diastole ED in each of three cardiac cycles obtained by real-time simulation are displayed in pair on the upper right part, and a corresponding cardiac cycle indication is displayed on the lower right part, wherein in the cardiac cycle indication, dark-colored dots are used to represent three end-diastoles ED, and light-colored dots are used to represent three end-systoles ES.

As shown in FIG. 9, when an examiner wants to select the image of end-diastole ED and the image of end-systole ES of the second set appearing in a top-down direction in FIG. 9 as images of a target cardiac cycle to examine the state of the heart, the image of end-diastole ED and the image of end-systole ES of the second set are selected in the display screen A1 by means of a selection box in response to an instruction from an input unit. Thus, the image of end-diastole ED and the image of end-systole ES of the second set are displayed in a highlighted manner.

At the same time, synchronously in response to the instruction, the cardiac cycle corresponding to the image of end-diastole ED and the end-systole ES of the second set in the cardiac cycle indication are displayed in a highlighted manner on the lower right part of the display screen A1, without displaying in a highlighted manner the remaining cardiac cycles.

Furthermore, as shown in FIG. 10, in the display screen A2 corresponding to step G above, on the basis of the display screen A1 shown in FIG. 9, when the examiner needs to select the image of end-diastole ED and the image of end-systole ES of the third set appearing in a top-down direction as images of a target cardiac cycle to examine the state of the heart, the examiner can input an instruction by manually sliding on the touch panel, rotating the knob, or pressing the keyboard, etc. In response to the instruction, in the display screen A2, the selection box changes from the state of selecting the image of end-diastole ED and the image of end-systole ES of the second set to a state of selecting the image of end-diastole ED and the image of end-systole ES of the third set. At the same time, in the cardiac cycle indication shown in the lower right part, the cardiac cycle corresponding to the image of end-diastole ED and the image of end-systole ES of the third set are also synchronously displayed in a highlighted manner, without displaying in a highlighted manner the remaining cardiac cycles.

As described above, the medical imaging method according to an embodiment of the present disclosure allows an examiner to select and display in a highlighted manner as needed, through an interactive user interface, an image of end-systole ES and an image of end-diastole ED as a set, and at the same time, a corresponding cardiac cycle is displayed in a highlighted manner in the cardiac cycle indication without displaying other cardiac cycles, thereby enhancing the convenience in heart disease diagnosis by the examiner.

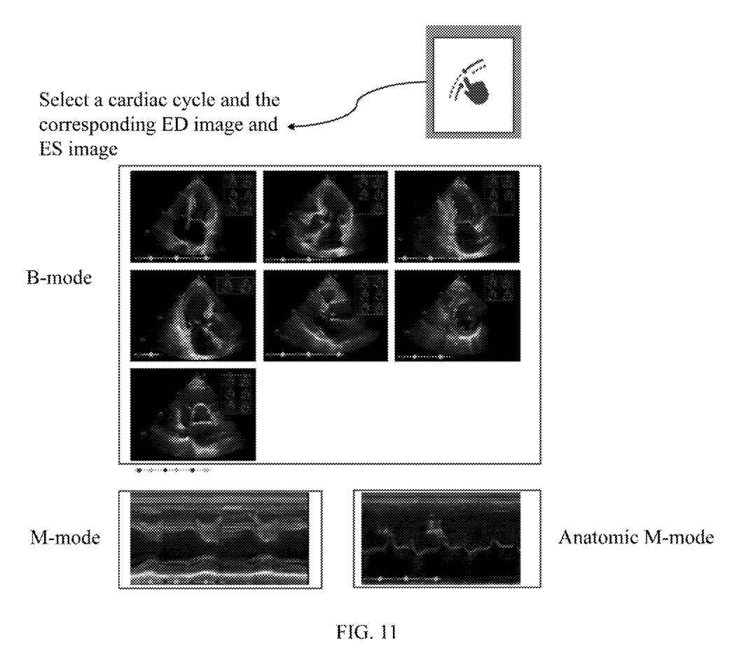

The beneficial effects of the medical imaging method according to an embodiment of the present disclosure will be described below with reference to FIG. 11 and on the basis of differences between the medical imaging method according to an embodiment of the present disclosure and the prior art.

FIG. 11 is a diagram showing a comparison between a case where a determined cardiac cycle is displayed in B-mode using the medical imaging method according to an embodiment of the present disclosure and a case where a determined cardiac cycle is displayed in M-mode or anatomic M-mode in the prior art.

As shown on the lower part of FIG. 11, the mode is switched to M-mode or AMM-mode, in which the cardiac cycle is determined and displayed using related time information.

Although the cardiac cycle can also be accurately determined by using related time information in M-mode or AMM-mode, since an image in two-dimensional mode obtained in B-mode cannot be used at the same time to observe the structure and state of the heart, a comprehensive view of the structure and function of the heart cannot be provided at the same time. As a result, an examiner often needs to switch between B-mode and M-mode or AMM-mode to the extent that it would be difficult for the examiner to accurately and quickly observe information of various structures and states of the heart. Moreover, the computational burden of the ultrasound device will be increased, which will result in decreased resolution of the ultrasound device.

By contrast, as shown on the upper part of FIG. 11, the case is shown where a cardiac cycle is determined by generating virtual sampling lines for a determined anatomical structure of interest in B-mode, without switching to M-mode or AMM-mode. Since cardiac cycles determined in the above manner are also displayed while an image in two-dimensional mode or a video in two-dimensional mode obtained using B-mode is displayed, and information related to one of the cardiac cycles can be selected and displayed in a highlighted manner in response to an instruction from an input unit such as a touch panel, a comprehensive view of the structure and function of the heart can be provided at the same time, which is convenient for the examiner to accurately and quickly observe information of various structures and states of the heart.

The medical imaging method according to an embodiment of the present disclosure has been hereto described. The medical imaging method innovatively introduces virtual cursor lines so as to transform analysis of an image in two-dimensional mode into analysis of an image in a different mode. As a result, the need to develop multiple models for different scanning views is avoided, and the detection of a cardiac cycle is implemented through a unified method, thereby greatly reducing system complexity, computational burden, and resource consumption.

According to an embodiment of the present disclosure, a computer-readable storage medium is further provided, having coded instructions recorded thereon, such that when the instructions are executed, the foregoing medical imaging method according to an embodiment of the present disclosure can be implemented. The computer-readable storage medium may include a hard disk drive, a floppy disk drive, a CD-read/write (CD-R/W) drive, a digital versatile disc (DVD) drive, a flash drive and/or a solid-state storage device.

According to an embodiment of the present disclosure, a medical imaging system is further provided, the medical imaging system comprising a processor configured to execute the foregoing medical imaging method according to an embodiment of the present disclosure.

The foregoing medical imaging system can implement the foregoing medical imaging method according to an embodiment of the present disclosure. Many design concepts and details described above which are applicable to the medical imaging method according to an embodiment of the present disclosure are also applicable to the foregoing medical imaging system, and can produce the same beneficial technical effects, which will not be repeated here.

Various aspects of the present disclosure have been described above via some exemplary embodiments. However, it should be understood that various modifications can be made to the exemplary embodiments described above without departing from the spirit and scope of the present disclosure. For example, an appropriate result can be achieved if the described techniques are executed in a different order and/or if the components of the described system, architecture, device, or circuit are combined in other manners and/or replaced or supplemented with additional components or equivalents thereof; accordingly, the modified other implementations also fall within the protection scope of the claims.

1. A medical imaging method, comprising:
performing acquisition on a section of the heart in chronological order to obtain multiple images in two-dimensional mode;
identifying at least one anatomical structure of interest in the multiple images in two-dimensional mode and determining a coordinate point of the at least one anatomical structure of interest;
generating at least one virtual sampling line in the multiple images in two-dimensional mode, the at least one virtual sampling line passing through the at least one anatomical structure of interest;
generating a displacement time map of the at least one anatomical structure of interest by using the at least one virtual sampling line based on the multiple images in two-dimensional mode; and
detecting multiple corresponding sets of end-systolic coordinates and end-diastolic coordinates on the basis of the displacement time map to determine multiple cardiac cycles.

2. The medical imaging method according to claim 1, further comprising displaying the multiple images in two-dimensional mode or a video in two-dimensional mode consisting of the multiple images in two-dimensional mode, and at the same time displaying an image of end-systole and an image of end-diastole in each of the multiple cardiac cycles and a cardiac cycle indication.

3. The medical imaging method according to claim 2, further comprising, in response to an instruction from an input unit, selecting one cardiac cycle from the multiple cardiac cycles as a target cardiac cycle, displaying in a highlighted manner the image of end-systole and the image of end-diastole corresponding to the target cardiac cycle, and displaying in a highlighted manner the target cardiac cycle in the cardiac cycle indication.

4. The medical imaging method according to claim 1, wherein the at least one virtual sampling line comprises multiple virtual sampling lines, and
the generating a displacement time map of the at least one anatomical structure of interest comprises:
using the coordinate point of the at least one anatomical structure of interest as a center point to generate the multiple virtual sampling lines passing through the center point; and
generating multiple displacement time maps as described on the basis of the multiple virtual sampling lines, and
the determining multiple cardiac cycles comprises:
stacking multiple displacement time maps as described along the Y-axis to obtain a second composite displacement time map; and
detecting multiple corresponding sets of the end-systolic coordinates and the end-diastolic coordinates on the basis of the second composite displacement time map to determine the multiple cardiac cycles.

5. The medical imaging method according to claim 1, wherein the at least one anatomical structure of interest comprises multiple anatomical structures of interest, and
the generating a displacement time map of the at least one anatomical structure of interest comprises:
generating the at least one virtual sampling line for each of the multiple anatomical structures of interest, respectively; and
generating multiple displacement time maps as described on the basis of the multiple generated virtual sampling lines as described, and
the determining multiple cardiac cycles comprises:
stacking multiple displacement time maps as described along the Y-axis to obtain a first composite displacement time map; and
detecting multiple corresponding sets of the end-systolic coordinates and the end-diastolic coordinates on the basis of the first composite displacement time map to determine the multiple cardiac cycles.

6. The medical imaging method according to claim 1, wherein the at least one anatomical structure of interest is identified using a first neural network model.

7. The medical imaging method according to claim 6, wherein the multiple cardiac cycles are determined using a second neural network model different from the first neural network model.

8. The medical imaging method according to claim 7, wherein the second neural network model only predicts an X-axis coordinate, and the multiple corresponding sets of the end-systolic coordinates and the end-diastolic coordinates are detected by processing the X-axis coordinate.

9. The medical imaging method according to claim 1, wherein the multiple images in two-dimensional mode are any one of an image in B-mode, an image in M-mode, or an image in anatomic M-mode.

10. The medical imaging method according to claim 1, wherein the section and the at least one anatomical structure of interest are selected from at least one of the following schemes:
the section is a two-chamber cardiac section, and the at least one anatomical structure of interest comprises a mitral valve;
the section is a three-chamber cardiac section, and the at least one anatomical structure of interest comprises a mitral valve or an aortic valve;
the section is a four-chamber cardiac section, and the at least one anatomical structure of interest comprises a mitral valve or a tricuspid valve;
the section is a five-chamber cardiac section, and the at least one anatomical structure of interest comprises a mitral valve and a tricuspid valve;
the section is a parasternal long-axis section, and the at least one anatomical structure of interest comprises a mitral valve or an aortic valve; or
the section is a short-axis section, and the at least one anatomical structure of interest comprises an aortic valve.

11. A medical imaging system, comprising:
a memory storing instructions; and
a processor configured to execute the instructions to:
perform acquisition on a section of the heart in chronological order to obtain multiple images in two-dimensional mode;
identify at least one anatomical structure of interest in the multiple images in two-dimensional mode and determining a coordinate point of the at least one anatomical structure of interest;
generate at least one virtual sampling line in the multiple images in two-dimensional mode, the at least one virtual sampling line passing through the at least one anatomical structure of interest;
generate a displacement time map of the at least one anatomical structure of interest by using the at least one virtual sampling line based on the multiple images in two-dimensional mode; and
detect multiple corresponding sets of end-systolic coordinates and end-diastolic coordinates on the basis of the displacement time map to determine multiple cardiac cycles.

12. A non-transitory computer readable medium storing instructions that, when executed by a computer, cause the computer to:
perform acquisition on a section of the heart in chronological order to obtain multiple images in two-dimensional mode;
identify at least one anatomical structure of interest in the multiple images in two-dimensional mode and determining a coordinate point of the at least one anatomical structure of interest;
generate at least one virtual sampling line in the multiple images in two-dimensional mode, the at least one virtual sampling line passing through the at least one anatomical structure of interest;
generate a displacement time map of the at least one anatomical structure of interest by using the at least one virtual sampling line based on the multiple images in two-dimensional mode; and
detect multiple corresponding sets of end-systolic coordinates and end-diastolic coordinates on the basis of the displacement time map to determine multiple cardiac cycles.
